(19) United States
(12) Patent Application Publication
(10) Pub. No.: US 2010/0143305 A1
(43) Pub. Date: Jun. 10, 2010

(54) TREATMENT OF HIV AND AIDS USING PROBIOTIC LACTOBACILLUS REUTERI

(76) Inventor: James Allen Lemke, Malone, WI (US)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 12/633,642

(22) Filed: Dec. 8, 2009

Related U.S. Application Data
(60) Provisional application No. 61/120,708, filed on Dec. 8, 2008.

(51) Int. Cl. A61K 35/74; A61P 31/18

(52) U.S. Cl. 424/93.2

(57) ABSTRACT

A method for treating or preventing a Human Immunodeficiency Virus (HIV) infection, or treating or preventing Acquired Human Immunodeficiency Syndrome (AIDS), in a subject in need thereof, is disclosed. The method involves colonizing a genetically modified probiotic Lactobacillus reuteri RC-14 in the gastrointestinal tract of a subject, wherein the probiotic is able to secrete two or more fusion inhibitors that decrease or prevent HIV production and CD4+T cell depletion in the gastrointestinal tract.

### FIELD OF THE INVENTION

The present invention relates generally to preventing the acquisition or progression of Human Immunodeficiency Virus (HIV) through probiotic treatment. Specifically, the invention relates to a method of reducing or preventing HIV replication in a cell, and/or reducing or preventing depletion of CD4+T cells.

### BACKGROUND OF THE INVENTION

Currently, many medications have been developed in an effort to slow the progression of HIV and slow the onset of Acquired Immunodeficiency Syndrome (AIDS). Most of these drugs are unable to keep up with HIV's high mutation rates. Consequently, in many cases, patients will develop drug resistance as the HIV will mutate its surface antigen frequently. To date, no vaccine or cure for HIV exists.

Recently, a newer class of anti-HIV drugs referred to as fusion inhibitors has been used in an attempt to protect CD4+T cells from viral infection. Enfuvirtide, or T20, is one of the only FDA-approved fusion inhibitors. However, a drawback to T20 is that it quickly develops drug resistance and must be administered subcutaneously (He et al., 2008, Proc. Natl. Acad. Sci. U.S.A. 105(42): 16332-7). Subcutaneous injection of anti-HIV drugs make administration to large groups of individuals long and difficult.

Another fusion inhibitor currently approved by the FDA is Maraviroc. Maraviroc is a CCR5 co-receptor antagonist that provides efficacy when other medications develop drug resistance (Emmelkamp et al., Expert Opinion on Drug Safety 7(5): 559-69). While the drug is administered in tablet form, making it easily dispensed to large groups of people quickly and efficiently, there are a few set backs to using Maraviroc as well. First, Maraviroc is only used for individuals whose latent HIV infection has mutated to become CCR5-tropic HIV-1. Tropism testing is required, and such testing is quite expensive. In addition, regular dose adaptations are required as there are declining numbers of patients with advanced HIV-1 of CCR5 tropism due to tropism-shifting and drug-drug interactions.

Two other HIV fusion inhibitors have been recently researched for their potential as treatment options. PSC-RANTES (Regulated upon Activation, Normal T-cell Expressed and Secreted), or CCL5, is a highly potent chemokine entry inhibitor against R5-tropic HIV-1 strains (Gaertner et al., Proc. Natl. Acad. of Sci. U.S.A. 105(46): 17706-11). PSC-RANTES demonstrates to be fully protective in the vaginal transmission of HIV in rhesus macaques. However, the chemical synthesis of PSC is expensive and can not be naturally synthesized. In addition, PCS-RANTES' strong CCR5 antagonistic properties lead to local inflammation.

Another inhibitor currently under investigation is Macrophage Inflammatory Protein 1β (MIP-1β). MIP-1β is the normal ligand for CCR5 and has recently been found to have potent HIV-1 infection inhibiting properties in peripheral blood mononuclear cells (PBMC) (Menten et al., J. Clin. Invest. 104: R1-R5 (1999); Nibbs et al., J. Biol. Chem. 274: 17478-17483 (1999)), monocytes, and macrophages (Aquaro et al., J. Virology. 275: 4402-06 (2001)). Though this HIV entry inhibitor is an effective molecule, it is also a CCR5 antagonist. Thus, it is possible that MIP-1β may activate granulocytes (neutrophils, eosinophils, and basophils) causing acute neutrophilic inflammation.

Current prophylactic treatment of HIV-negative patients is most effective while using antiretroviral (ARV) drugs. Two Pre-exposure prophylactic (PrEP) drugs are currently being researched; tenofovir disoproxil fumarate (TDF) and TDF+emtricitabine (FTC). PrEPs have a favorable resistance profile, possibly to protect mother-to-infant transmission, and are safe in humans. ARVs can be taken orally and remain in the blood for extended periods of time, but ARVs do not deliver a prophylactic drug dose directly to the site of greatest initial viral replication and CD4+T cell depletion, i.e. the gut-associated lymphatic tissue.

Recently, research has indicated that Lactobacillus reuteri ATCC 55730 can successfully colonize the human gastrointestinal tract when administered orally (Valeur, N. et al., 2004, Applied and Environmental Microbiology, 70(2): 1176-1181). The research also indicates that healthy subjects tolerate daily doses of Lactobacillus reuteri ATCC 55730 totaling 4×10⁸ CFU/day well, only complaining of slight flatus during the course of administration. A significant increase in live Lactobacillus reuteri ATCC 55730 is found in fecal samples. This study establishes colonization of this probiotic in the stomach and duodenum, and other studies report the successful colonization of a lactobacillus species in the jejunum and rectum of humans (Johansson, M. L. et al., 1993, Appl. Environ. Microbiol. 59:15-20).

Furthermore, several studies indicate that Lactobacillus reuteri may be able to modulate the immune system in the gastrointestinal tract (Valeur, N. et al., 2004, Applied and Environmental Microbiology, 70(2): 1176-1181; Christensen, H. R. et al., 2002, J. Immunol., 168:171-178; Maasen, C. B. et al., 200, Vaccine, 18:2613-2623; Madsen, K. L. et al., 1999, Gastroenterology, 116:1107-1114; Tejada-Simon, M. V. et al., 1999, J. Food Prot., 62:1435-1444; Tejada-Simon, M. V. et al., 1999, J. Food Prot., 62:162-69). Most notably, studies indicate an increase in CD4+T cell activation in the lamina propria of the small intestine while lactobacillus species are present in the human gut (Ferreira, R. et al., 1990, Gastroenterology, 98:1255-1263; Banasaz, M., 2002, Appl. Environ. Microbiol., 68:3031-3034), including Lactobacillus reuteri ATCC 55730 (Casas, I. A. et al., 2000, Microb. Ecol. Health Dis., 12:247-285)

Accordingly, an inexpensive yet effective therapeutic and prophylactic treatment for HIV is desired. The treatment should be easily administered to those who are susceptible to or already have the virus. Additionally, the administration method for such a treatment should be such that a continuous supply of anti-viral molecules could be delivered in direct proximity to the site of the largest viral replication. The present invention addresses these issues, as well as the problems presented in the prior art.

### SUMMARY OF THE INVENTION

In light of the foregoing, it is an object of the present invention to provide a method for treating or preventing a Human Immunodeficiency Virus (HIV) infection, or treating or preventing Acquired Human Immunodeficiency Syndrome (AIDS), in a subject in need thereof, comprising colonizing a genetically modified probiotic Lactobacillus reuteri RC-14 in the gastrointestinal tract of a subject, the probiotic Lactobacillus reuteri RC-14 having the ability to secrete two or more fusion inhibitors; and decreasing or preventing HIV production and CD4+T cell depletion in the gastrointestinal tract because of the presence of the two or more fusion inhibitors. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It can further be an object of the instant invention to provide a method of reducing or preventing HIV replication in a CD4+T cell in gut associated lymphatic tissue (GALT) of a gastrointestinal tract, comprising interfering with the binding, fusion or entry of HIV viron into the CD4+T cell in GALT by two or more fusion inhibitors secreted by a genetically modified probiotic Lactobacillus reuteri RC-14. In an aspect of the invention, two or more stages of HIV viral entry are reduced or blocked.

It is another object of the present invention to provide a method of reducing or preventing depletion of CD4+T cells in GALT of a gastrointestinal tract caused by initial HIV infection, comprising reducing or preventing HIV replication in the GALT by presence of two or more fusion inhibitors secreted by a genetically modified probiotic Lactobacillus reuteri RC-14.

Other objects, features, benefits and advantages of the present invention will be apparent from this summary and the following descriptions of certain embodiments, and will be readily apparent to those skilled in the art. Such objects, features, benefits and advantages will be apparent from the above as taken into conjunction with the accompanying examples, data, and all reasonable inferences to be drawn therefrom.

### BRIEF DESCRIPTION OF THE DRAWINGS

FIG. 1 shows the three sites of viral entry where PRO 542, MIP-1β and T-1249 bind.

FIG. 2 shows the results obtained from analyzing the affects of SIV viral infection on CD4+T cell levels.

FIG. 3 displays the results from the immunostaining of Lactobacillus reuteri ATCC 55730 obtained from human gut colonies.

FIG. 4 shows densitometry results and the ability of Lactobacillus reuteri RC-14 to secrete HIV entry and fusion inhibitors.

### DETAILED DESCRIPTION OF THE INVENTION

Illustrating certain non-limiting aspects and embodiments of this invention, a method for treating or preventing a Human Immunodeficiency Virus (HIV) infection, or treating or preventing Acquired Human Immunodeficiency Syndrome (AIDS), in a subject in need thereof, is disclosed. The method involves engineering Lactobacillus reuteri RC-14 to secrete at least two fusion inhibitors, colonizing the engineered probiotic Lactobacillus reuteri RC-14 in the gastrointestinal tract of a subject, secreting the fusion inhibitors in the gastrointestinal tract, and decreasing or preventing HIV production and CD4+T cell depletion in the gastrointestinal tract because of the presence of the two or more fusion inhibitors.

In an embodiment of the invention, one of the two or more fusion inhibitors inhibit the function of CCR5, a beta chemokine receptor found on the surface of CD4+T cells. MIP-1β is a non-limiting example of such an inhibitor. An advantage of inhibiting CCR5 is that blocking its function appears to have no effect on normal immune functioning, and it has even been seen in certain populations with CCR5 polymorphism as an immune adaptation (Salem, A. H. et al., 2007, Mutat. Res. 616(1-2): 175-80; De Silva, E. et al., 2004, 241(1): 1-12; Galvani, A. P. et al., 2005, 7(2): 302-309). In these studies, a rare 32-basepair (bp) deletion, known as the CCR5-Δ32 mutation, found in the allele of the beta-chemokine receptor gene CCR5, is shown to prevent cellular invasion by HIV-1 strains in individuals homozygous (Δ32/Δ32) for the deletion. Essentially, individuals with the CCR5-Δ32 deletion fail to express functional CCR5. Homozygous Δ32 individuals are not completely resistant to contracting the HIV-1 strain, as it is suspected that dual tropic strains of HIV-1 utilize the CXCR4 co-receptor to gain entry into cells. However, this study demonstrates that individuals with defective CCR5 still have normally functioning immune systems, and blocking CCR5 does not compromise immune function. Thus, CCR5 fusion inhibitors are a viable option for blocking HIV entry into CD4+T cells.

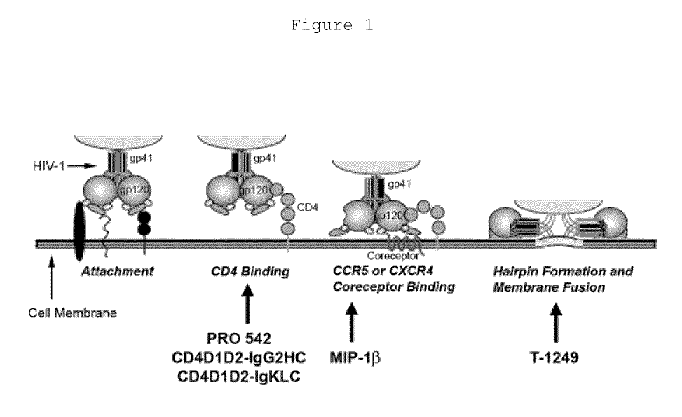

MIP-1β is the normal ligand for CCR5 which activates granulocytes to initiate acute neutrophilic inflammation (FIG. 1) (Liu, J. J. et al., 2007, Cellular Microbiology, 9: 120-130), but also displays the most potent fusion inhibition of HIV-1 infection among other chemokines in PBMCs, monocytes, and macrophages (Liu, J. J. et al., 2007, Cellular Microbiology, 9: 120-130; Menten, P. et al., 1999, J. Clin. Invest., 104: R1-R5; Nibbs, R. J. et al., 1999, J. Biol. Chem., 274: 17478-17483; Aquaro, S. et al., 2001, J. Virology., 75: 4402-06). The second stage of HIV-1 entry occurs through the interaction of viral gp120 and CCR5, found on T cells and macrophages (Liu, J. J. et al., 2007, Cellular Microbiology, 9: 120-130). MIP-1β is capable of providing a second line of defense against viral entry through its potent entry fusion inhibition properties as a CCR5 antagonist. MIP-1β is especially useful as a fusion inhibitor as it is naturally synthesized, not recognized by the body as foreign, and its function in blocking CCR5 does not compromise the normal functioning of the immune system (Blanpain, C. et al., 2002, Receptors Channels, 1:19-31).

In yet another embodiment of the invention, one of the two or more fusion inhibitors can block binding to CD4. PRO 542, a recombinant antibody-like fusion protein, is a non-limiting example of such an inhibitor. The D1D2 domain of human CD4 replaces the heavy- and light-chain variable domains of human IgG2 in PRO 542 (FIG. 1) (Liu, J. J. et al., 2007, Cellular Microbiology, 9: 120-130; Allaway, G. P. et al., 1995, AIDS Res. Hum. Retroviruses, 11: 533-39; Zhu, P. et al., 2001, J. Virology, 75: 6682-86), thus preventing the attachment of the HIV-1 surface glycoprotein gp120 to CD4+T cells. As such, PRO 542 is proven to be effective in phase II clinical trials of HIV-positive patients when other treatment options fail (Liu, J. J. et al., 2007, Cellular Microbiology, 9: 120-130; Jacobson, J. M. et al., 2004, 48: 423-429).

In still another embodiment of the invention, one of the two or more fusion inhibitors is involved with hairpin formation and membrane fusion mechanism. A non-limiting example of such an inhibitor is T-1249. T-1249 is a peptide fusion inhibitor, much like T-20, that remains active as a fusion inhibitor even when the HIV-1 strain develops T-20 resistance (FIG. 1) (Liu, J. J. et al., 2007, Cellular Microbiology, 9: 120-130; LaBranche, C. C. et al., 2001, Expert Opin. Invest. Drugs, 9: 371-382). T-1249 inhibits the third stage of viral entry, which involves the spring-loaded action of viral gp41 and hairpin formation which allows viral membrane fusion of the targeted cell. In vitro studies indicate T-1249 is 10-fold more potent than T-20 (Conway, B., 2000, Curr. Opin. Anti-Infect Invest Drugs, 2: 317-322), and is found to be potent against HIV-1 infection in HIV-positive adults after a 14 day phase 1-2 dose escalation study (Liu, J. J. et al., 2007, Cellular Microbiology, 9: 120-130; Eron, J. J. et al., 2004, J. Infect Dis., 189: 1075-1083). A more recent study in rhesus macaques reveals that T-1249 is potent as a tropism-independent HIV-1 inhibitor, as well (Veazey R. S. et al., 2008, Proc. Natl. Acad. Sci. U.S.A., 105(30): 10531-6).

In another embodiment of the invention, PRO 542, MIP-1β and T-1249 can all be used as fusion inhibitors. Using a combination of PRO 542, MIP-1β, and T-1249 provides fusion inhibitors at all three stages of the HIV entry process. As such, the likelihood of drug resistance becomes significantly less and the efficacy increases.

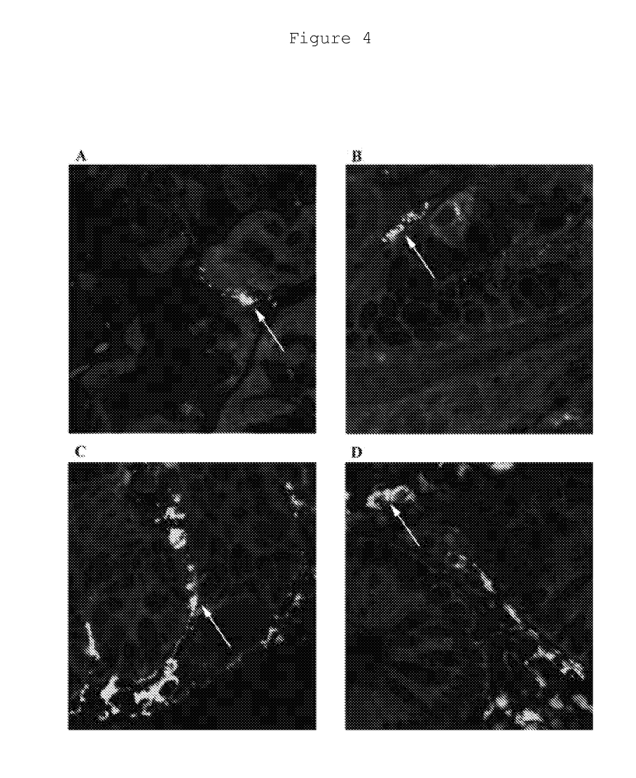

Liu et al. demonstrate the ability of Lactobacillus reuteri RC-14 to secrete HIV entry and fusion inhibitors (FIG. 4) in the human vaginal tract (Liu, J. J. et al., 2007, Cellular Microbiology, 9: 120-130). Three gene fragments (BspA, MIp, and Sep) are inserted into the chromosome of Lactobacillus reuteri RC-14. These three fragments are encoded to translate the fusion inhibitors PRO 542, MIP-1β, and T-1249, respectively.

Western blots using monoclonal anti-human CD4 or anti-6 histidine (6 hi) tag antibodies are performed to determine if Lactobacillus reuteri RC-14 expresses these three recombinant protein and peptide fusion inhibitors. All recombinant Lactobacillus reuteri are able to secrete both cell wall associated and secreted recombinant proteins and peptides. However, it was discovered that full-length BspA fusion inhibitors are more likely to be cell wall associated while MIp and Sep fusion protein are more likely to be secreted or trapped in the cell wall during export.

The expression levels of each recombinant protein produced by the recombinant DNA is analyzed by densitometry (FIG. 4b, 4c). The concentration results for every recombinant protein are shown in FIGS. 4b and 4c.

In a preferred embodiment, the method further comprises oral administration of the engineered Lactobacillus reuteri RC-14 resulting in the colonization of this probiotic to the GI tract. As such, secretion of the fusion inhibitors is proximate to the largest site of initial viral replication and CD4+T cell depletion in the body, i.e. the GALT. Oral administration is advantageous as it can be administered to large groups of individuals who are susceptible to contracting the virus. Thus, a continuous supply of anti-viral molecules is delivered in a non-invasive manner. Administration is quick and easy, and can be taken without intervention on a daily basis.

Unlike the ARV PrEPs which persist in the blood, the use of this probiotic prophylactic treatment process for individuals at high risk of contracting HIV-1 virus provides a direct and constant source of fusion inhibitors delivered to the site of greatest initial viral replication and CD4+T cell destruction. Furthermore, the treatment delivers three potent fusion inhibitors which inhibit viral entry during three different stages of HIV-1 cellular entry. This greatly decreases the likelihood of the virus developing drug resistance through mutation. This also decreases the expensive screening for HIV-tropism, as this drug is not reliant on the tropism of HIV strain. In addition, this drug would be quickly and easily administered orally to large groups of individuals who are at high risk of contracting strains of HIV.

Also, once the Lactobacillus is engineered, copies are easily grown, replicated, stored, and transported. Using bacteria to synthesize these protein fusion inhibitors is also far less expensive than chemically synthesizing inhibitory molecules, such as PSC-RANTES. In addition, Lactobacillus is able to colonize the human gastrointestinal tract with little to no side effects and is also known to increase CD4+T cells levels in the lamina propria of the GALT.

By colonizing the engineered Lactobacillus in the gut, the approach provides a direct and constant supply of HIV fusion inhibitory molecules to the GALT. Doing this establishes a potent defense against HIV replication and CD4+T cell depletion in the GALT during early HIV infection. By using this easily administered, inexpensive treatment, it may be possible to use engineered Lactobacillus to prevent preliminary HIV infection for large groups of individuals who are susceptible to contracting the virus.

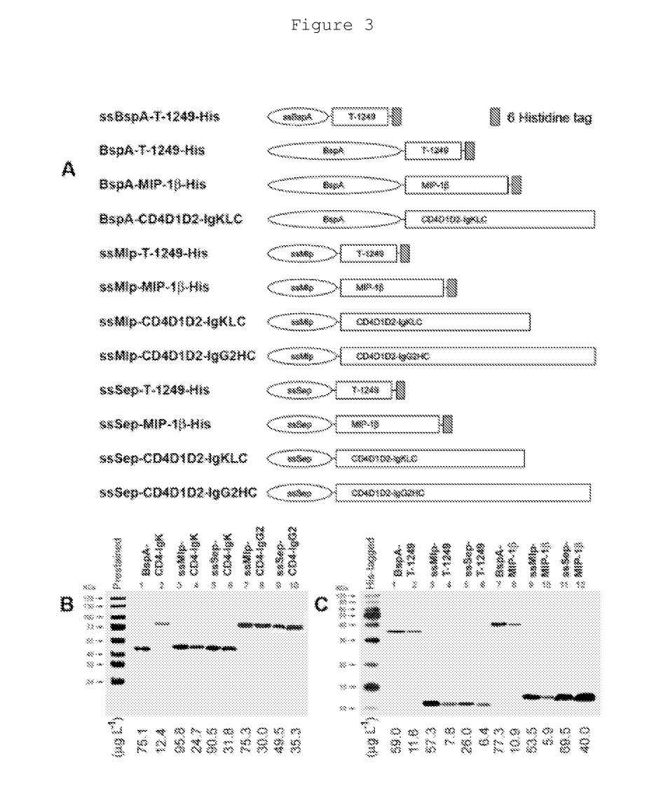

Lactobacillus reuteri ATCC 55730 is a probiotic bacterium commonly found in dietary supplements (Valeur, N. et al., 2004, Applied and Environmental Microbiology, 70(2): 1176-1181). Valeur et al. demonstrate Lactobacillus reuteri ATCC 55730′ s ability to colonize the gastrointestinal tract of humans (FIG. 3) with little to no change in the bacterial DNA from the administered probiotic.

As such, in another embodiment of the invention, the depletion of CD4+T cells in GALT of a gastrointestinal tract caused by initial HIV infection is prevented or significantly reduced. The depletion of CD4+T cells in the GALT of rhesus macaques upon initial HIV/SIV pathogenesis is reported by Li et al. (Li, Q. et al., 2005, Nature, 434: 1148-1152), though cells located in the periphery of the body diminish at much slower rates (Veazey, R. S. et al., 1998, Science, 280: 427-431; Brenchley, J. M. et al., 2004, J. Exp. Med., 200: 749-759; Mattapallil, J. J. et al., 2005, Nature, 434: 1093-1097; Mehandru, S. et al., 2004, J. Exp. Med., 200: 761-770).

The GALT is the largest component of the lymphoid system and the lamina propria of the GALT is the main site of viral production and CD4+T cell depletion for two likely reasons: (1) the GALT is comprised of most (−60%) of the body's secondary lymphoid organs, and (2) the GALT hosts a large number of recently activated (due to antigenic stimulation) CD4+T cells which remain in a resting-like state, allowing them to easily become infected with SIV.

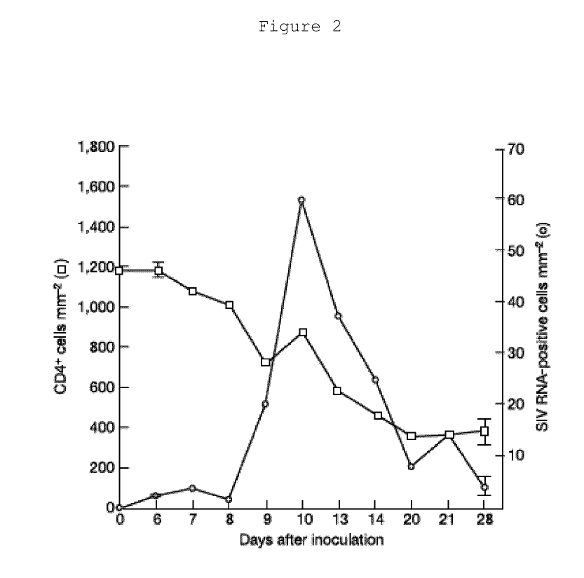

The GALT is also indicated as the primary site for initial SIV viral replication in the rhesus macaques, as reported by Li et al. The study focuses on the colon of rhesus macaques to represent the effects of SIV on the GALT and measures viral production copies of SIV RNA per microgram of tissue RNA. Virus production and CD4+T cell depletion is monitored for 28 days after inoculation. The viral peak of production occurs at 10 days after inoculation and coincides with the peak in SIV-RNA positive cells (FIG. 2). Though peak SIV-RNA positive cells are found in both the follicular inductive and diffuse effector arms of the GALT, the most massive depletion of CD4+T cells takes place in the lamina propria. CD4+T cell depletion in the lamina propria is detectable just six days after inoculation, but depletion rapidly accelerates between 8 and 14 days after inoculation (FIG. 2). The rate of depletion finally hits a plateau after day 14 at 70% below the initial CD4+T cell baseline (FIG. 2). Apparently, these results correspond to the selective loss of the entire lamina propria CD45RO⁺ and CD4+T cell population.

Li et al. also suggests that the majority of the SIV-RNA positive cells are in fact memory CD4+T cells. By detecting markers of early T cell activation, using CD69 (Veazey, R. et. al., 2000, J. Virology, 74: 57-64; Zhang, Z. Q., et. al., 1999, Science, 286: 1353-57; Zhang, Z. Q., et. al., 2004, Proc. Natl. Acad. Sci. USA., 101: 5640-45), and markers detecting sustained T cell activation and expression, using CD25 and Ki67, it is determined that 91-93% of SIV-RNA positive cells in the GALT at the peak of SIV-RNA positive cells in the study express the CD69⁻, CD25⁻, and Ki67⁻ phenotype. Unlike most cells initially infected in cervico-vaginal mucosa at peripheral lymph nodes, the markers of these cells show neither activation nor proliferation of cells. Therefore, most of the cells initially infected with SIV (cells positive for SIV-RNA) are memory cells that are depleted due to apoptosis by CD8+T cells.

Also, a 20 fold decrease in SIV-RNA positive cells from the viral peak at 10 days to the final day of monitoring at 28 days is shown. This is due to substrate exhaustion of the pool of resting T cells and the destruction of resting memory CD4+T cells that are not replaced after the initial infection. However, this is significant to the proposed treatment as it indicates activated, resting T cells substantially contribute to peak virus production in the GALT and ultimately the elimination of GALT CD4+T cells.

Though SIV-infected CD4+T cells during initial infection are found throughout the GALT of rhesus macaques, including both inductive and effector sites, the most severe depletions occur in the LPL and IEL. These studies demonstrate that initial viral replication and CD4+T cell depletion occur in specific portions of the GALT of rhesus macaques. The studies also prove rhesus macaques serve as an important model in which viral replication and CD4+T cell can be monitored. As such, rhesus macaque models are employed in the instant invention as they are believed to be important in establishing normal viral replication and CD4+T cell depletion upon SIV infection, and are believed to be important in the effects the engineered probiotic Lactobacillus have on potentially slowing or preventing both viral replication and CD4+T cells depletion in the GALT.

Further, rhesus macaques have inductive and effector sites located in their gastrointestinal tract. Inductive sites are sites where the immune response is initiated and antigen-presenting cells activate CD4+T cells. Effector sites are sites where CD4+T cells migrate to after activation. These sites are composed of different tissues and lymphocyte organization. Effector sites consist of intestinal epithelial lymphocytes (IEL) and lamina propria lymphocytes (LPL) (Mowat, A. M., 2003, Nat. Rev. Immunology, 3: 331-341). In rhesus macaques, the LPL is of greater interest in the instant invention as the proportion of activated CD4+T cells is higher here than in then peripheral blood or lymph nodes (Veazey, R. S. et al., 1998, Science, 280: 427-431; Veazey, R. S. et al., 1997, Clin Immunol Immunopathol, 82: 230-242).

Likewise, CD4+T cells are more prominent in Peyer's Patcher found in the ileum of rhesus macaques. These sites are included as inductive sites, and specialize by sampling antigen in the lumen through M cells. Such activity takes place in the GALT of rhesus macaques, especially in the lamina propria, and these excess levels of CD4+T cells are a major site of HIV replication and CD4+T cell depletion upon infection with HIV. In the case of rhesus macaques, the same is true when infected with SIV.

The disclosures in this application of all articles and references, including patents, are incorporated herein by reference. The invention is illustrated further by the following examples which are not to be construed as limiting the invention in scope or spirit to the specific procedures described herein. The starting materials and various intermediates may be obtained from commercial sources, prepared from commercially available compounds, or prepared using well known synthetic methods.

### EXAMPLES

### Example 1

Colonization of Engineered Lactobacillus reuteri in the Gut of Rhesus Macaques.

a) Selection of rhesus macaques as the animal model. The use of rhesus macaques (Macaca mulatta) as the animal model is desired. Rhesus macaques share a similar gastrointestinal tract as humans, and it has been demonstrated that Lactobacillus reuteri will colonize the gastrointestinal tracts of infant rhesus macaques safely and effectively (Kelleher S. L. et al., 2002, Pediatric Gastroenterology and Nutrition, 35(2):162-8). The use of a mouse model to examine the effects of HIV-1 on the GALT was considered, but a previous study using humanized mouse intestine indicated that although many aspects of HIV-1 GALT pathogenesis are recapitulated in these mice, it was not determined whether or not there is a direct and/or indirect pathological effect of HIV-1 on enterocytes, as seen in humans (Denton, P. W. et al., PloS Medicine, 5(1) 0079-0089). To create statistically reliable results in measuring the effectiveness of colonizing the engineered Lactobacillus reuteri in gastrointestinal tracts of rhesus macaques, four (4) adult female rhesus macaques are used in the treatment group. To ensure that inadvertent isolation of any Lactobacillus species naturally resistant to penicillin and aminoglycosides from the animals does not occur, a control group of four (4) adult female rhesus macaques is used to sample normal gut flora and isolate natural Lactobacillus species.

b) Engineering of Lactobacillus reuteri with plasmid for aminoglycoside and penicillin resistance. Lactobacillus reuteri displays a large variety of resistance to antibiotics, especially vancomycin (Forbes, B. A. et al., 2002, Bailey and Scott's Diagnostic Microbiology, Eleventh Edition. Mosby, St. Louis, Mo.). A combination of aminoglycosides and penicillin is required for bactericidal activity against Lactobacillus species (Murray, P. R., Rosenthal, K. S., Pfaller, M. A., 2005, Medical Microbiology, Fifth Edition. Elsevier Mosby, Philadelphia, Pa.). Though other antibiotics elicit a bacteriostatic effect on Lactobacillus species, current research suggests that probiotic Lactobacillus species in the gut may transfer plasmids with antibiotic resistance to antibiotics targeting beta-lactam, tetracycline, and lincosamide (Rosander, A. et al., 2008, Applied and Environmental Microbiology, 74(19) 6032-40). To ensure that the natural plasmid transfer of antibiotic resistance that occurs in the gastrointestinal tract does not lead to false positive results of Lactobacillus reuteri colonization in rhesus macaques, inserting a plasmid with resistance that naturally occurring Lactobacillus species have no natural or acquired resistance is performed. In doing so, a bacteria is selected based on its resistance to aminoglycosides and penicillin.

To acquire such resistance, plasmids from Aldevron with an inserted RmtC is obtained because they have a sequence known to demonstrate extraordinarily high level resistance against various aminoglycosides (Wachino, J. et al., 2006, Antimicrobial Agents and Chemotherapy, 50(1) 178-184). These plasmids also contain a sequence known for penicillin resistance, pdcA (Kimura, Y. et al., 1999, American Society of Microbiology, 181(15): 4696-99). The plasmid is inserted into Escherichia coli strain DH5α (Paustian, T. et al., 2007, Microbiology 304, General Microbiology Lab Manual).

To isolate the plasmid from E. coli strain DH5α, the isolation technique outlined by Paustian et al. is followed. 2 ml of the DH5a culture and centrifuge for 15 seconds is taken. 0.1 ml of lysozyme-glucose solution is added along with 0.2 ml of Alkaline-SDS, 0.15 ml of High Salt solution, and 0.9 ml of ice cold ethanol. The mixture is subsequently centrifuged again for 5 minutes and the supernatant is removed. The pellet is resuspended in 0.1 ml of Acetate-MOPS and re-precipitated by adding 0.4 ml of 95% ethanol. After another 5 minutes of centrifugation, 40 μl of EDTA-Tris is added to resuspend the pellet.

The success of plasmid isolation is determined through PCR amplification. To accomplish this, portions of the plasmid is sequenced using sequence specific primers for the plasmid purchased, as well as restriction enzymes specific for this plasmid. This allows for the purity of the isolated plasmid to be determined. The plasmid is then transformed into Lactobacillus reuteri RC-14 through electrotransformation according to Turner et al. (Turner, M. S. et al., 1999, J. of Bacteriology, 181: 2192-2198) using 2.5 μg/ml of penicillin G as a cell wall weakening agent. The Lactobacillus is then incubated at 30° C. or 40° C. and 5% CO₂ for 3 to 4 days after sublethal incubation at 30° C. and 5% CO₂ overnight.

c) Plasmid transfer. To test the success of the plasmid transfer, the engineered Lactobacillus species is plated in LBS broth after incubation at 37° C. for 48 hours, followed by transfer to prereduced, anaerobically sterilized (PRAS) media supplied by Anaerobic Systems of Morgan Hill, California (Forbes, B. A. et al., 2002, Bailey and Scott's Diagnostic Microbiology, Eleventh Edition. Mosby, St. Louis, Mo.). This media supports the growth of anaerobic bacteria, hence the remaining Lactobacillus species. Small disks soaked in penicillin and aminoglycosides are placed on the PRAS media. The bacteria that grows on this agar is the engineered bacteria, as it is selected from other Lactobacillus through its acquired penicillin and aminoglycoside resistance.

d) Administration of engineered Lactobacillus reuteri in rhesus macaques. The microencapsulated Lactobacillus reuteri from c) is administered orally twice daily to the four rhesus macaques in the treatment group (Turner, M. S., 2003, Applied and Environmental Microbiology, 69: 5855-5863). The Lactobacillus reuteri is encapsulated in the biological polymer, alginate, as it is known to enhance the survival of anaerobic bacteria administered orally to the gastrointestinal tract, and fed to the monkeys. On average, the female rhesus macaque receives two daily doses of 2.62×10⁶ CFU Lactobacillus reuteri RC-14 per kg of body weight.

To ensure adequate colonization in the gut of the rhesus macaques, two separate doses of Lactobacillus reuteri RC-14 is administered to different rhesus macaques. The first dose is administered at the regular 2.62×10⁶ CFU Lactobacillus reuteri RC-14 per kg body weight. The second dose is administered at 1.5 times the regular dose, or roughly 4.0×10⁶ CFU Lactobacillus reuteri RC-14 per kg. If neither dose yields a successful colonization, doses 2 and 0.5 times the regular dose is administered. The dose is continued to be adjusted in a similar fashion until an appropriate dose for administration is found.

e) Collection and transport of gastrointestinal bacteria sample. After 28 days, bacterial samples are acquired from the gastrointestinal tract from the treatment group of rhesus macaques to determine whether or not the engineered Lactobacillus reuteri colonized. Bacterial samples from the control group of rhesus macaques is acquired to ensure the natural Lactobacillus species in the monkey gut does exhibit natural penicillin and aminoglycoside resistance. In general, material for anaerobic cultures, such as Lactobacillus species, is best obtained by aspiration using a needle and syringe.

f) Isolation and Identification of the engineered Lactobacillus reuteri. The specimens are stored in GasPak anaerobic jars during processing and examination of cultures, and isolation and identification is performed in an anaerobic chamber. The chamber will maintain an anaerobic environment with a gas mixture of 5% CO₂, 10% hydrogen, and 85% nitrogen.

Lactobacillus species is isolated from the rest of the aspirate sample by placing a portion of the sample in Lactobacillus Selection Broth (LBS) from BD Diagnostic Systems. The bacteria that remains from the LBS broth after incubation at 37° C. for 48 hours is transferred to PRAS media supplied by Anaerobic Systems of Morgan Hill, California. This media supports the growth of anaerobic bacteria, hence the remaining Lactobacillus species. To select the engineered strain of Lactobacillus reuteri, a solution of aminoglycoside and penicillin is mixed with top layer of the PRAS media. The plates are incubated at 37° C. for 48 hours. For the treatment group, the Lactobacillus that survives on this plate is the engineered Lactobacillus reuteri with the plasmid containing resistance to aminoglycosides and penicillin.

### Example 2

Secretion of PRO 542, MIP-1β, and T-1249 Lactobacillus reuteri RC-14 in the Gut of Rhesus Macaques.

a) The procedure for engineering Lactobacillus reuteri RC-14 to secrete PRO 542, MIP-1β, and T-1249 is performed according to Liu et al. (Liu, J. J. et al., 2007, Cellular Microbiology, 9: 120-130), in which a plasmid containing the gene fragments BspA, MIp, and Sep is transferred to Lactobacillus reuteri RC-14. The steps include as follows.

b) Culture of Lactobacillus reuteri RC-14 and preparation of genomic DNA. Wild-type and recombinant Lactobacillus reuteri is cultured in MRS broth or agar (bioWorld) without and with 10 μg per ml erythromycin at 37° C. and 5% CO₂, respectively. The strains are cultured in 5 ml of MRS broth to late stationary phase and collected by centrifuge at 3500 rpm. The collected strains are washed once with 10 mM Tris-HCl buffer including 1 mM EDTA (TE, pH=8) and lysed in the TE buffer including 10 mg per ml of lysozyme at 37° C. for 2 hours. The lysate is washed with proteinase K (1.5 mg ml⁻¹) and SDS (1.7%) and then incubated at 65° C. for 10 minutes. The genomic DNA is purified by extractions, once with Tris-saturated phenol and once with phenol-chloroform, and then precipitated with ethanol and dissolved in ultra-pure water at 65° C. for 3 minutes.

c) Sequencing of BspA, MIp, and Sep gene fragments in Lactobacillus reuteri RC-14. The genomic DNA of Lactobacillus reuteri is used to perform PCR using two pair of primers described below and advantage 2 polymerase, following the company protocol (Clontech). For sequencing of BspA gene fragments, the primers are designed according to published BspA gene sequence of L. fermentum BR11 (gi:2108225). The forward primer1 5′-TATTGCTGACTTGCAACAAGCAC-3′ and forward primer2, respectively, are paired with the reverse primer 5′-TTCTTCTTGCTGTAAGCGTACC-3′ to produce two cDNA fragments of BspA. The cDNA fragments are cloned into PCRII-TOPO vector using TOPO TA cloning kit (Invitrogen) and auto-sequenced in ABI 3730XL DNA sequencer using T7 or SP6 primers. For sequencing of MIp gene fragment, the primers are designed according to published MIp gene sequence of L. fermentum BR11 (gi:32395946). The forward primer is 5′-TTCGTCAATAAAGCCTAACAAGG-3′ and th reverse primer is 5′-CTAATACGGTAGTCACTAATAGC-3′. For sequencing of Sep gene fragment, the primers are designed according to published Sep gene sequence of L. fermentum BR11 (gi:45181527). The forward primer is 5′-GTAACCTTCCTGCTGACCT-3′ and the reverse primer is 5′-CAACACCCTATATACTACACTC-3′. The MIp and Sep PCR products are purified using QIAquick PCR purification kit (QIAGEN) and directly auto-sequenced in ABI 3730XL DNA sequencer with the primers described above. The similarity of three genes in L. fermentum BR11 and human Lactobacillus reuteri RC-14 are analyzed using BLAST program (2).

d) Cloning. All PCR products are cloned into PCRII-TOPO vector using TOPO TA cloning kit (Invitrogen) and auto-sequenced in ABI 3730XL DNA sequencer using T7 or SP6 primers to confirm the correction of sequences. PBMCs are prepared by the standard method and infected with laboratory adapted HIV-1 Bal virus. The infected cells are used to prepare genomic DNA intergrated with HVI-1 Bal provirus using QIAGEN RNA/DNA mini kit (QIAGEN). The resulting genomic DNA is utilized to perform PCR to create T-1249 cDNA fragments with the forward primer 5′-GGCTGCAG(Pstl)GAACTGCCTCAGCATGGCAAGAGTGGGAGCAAAAGATATACAG CTTAATTGAAGAATCGC-3′ and the reverse primer 5′-GCCTCGAG (Xhol)TTAG TGATGATGGTGATGATG(6 histidine tag)AAACCAATTCCACAAACTTGCCC-3′. The cDNA fragment of MIP-1β is generated by PCR with the forward primer 5′-GCCTGCAG(Pstl)GAACTGCCTCAGCA TCAGCACCAATGGGCTCAGACC-3′ and the reverse primer 5′-GGCTCGAG(Xhol)T TAGTGATGATGGTGATGATG(6 histidine tag) GTTCAGTTCCAGGTCATACACG-3′ using pET32a-MIP-1β plasmid as a template (NIH AIDS Research and Reference Reagent Program). The D1 and D2 domain of human CD4 (CD4D1D2) is produced through PCR with the forward primer 5′-GGCTGCAG(Pstl)GAACTGCCTCAGCAATGAACC GGGGAGTCCCTTTTAGG-3′ and the reverse primer 5′-CTGGAAAGCTAGC(Nehl)ACCACGATGTC-3′ using pSP65-CD4 plasmid as a template (NIH AIDS Research and Reference Reagent Program). Human CEMx174 cells (NIH AIDS Research and Reference Reagent Program) are cultured in RPMI 1640 medium with 10% FBS at 37° C. and 5% CO₂. Total cellular RNA of the cells are isolated using TRIzol reagent following the company protocol (Invitrogen). The cDNA is synthesized using oligo(dT) primer (Promega) and superscript II reverse transcriptase (Invitrogen) following the company protocol. The cDNA is used to perform PCR to generate the constant region of immunoglobulin gamma 2 heavy chain (IgG2HC) and the constant region of immunoglobulin kappa light chain (IgKLC) with the forward primer 5′-GGGCTAGC(Nhel)TTTCGAGCCAAATGTTGTGTCGAGTGC-3′ and the reverse primer 5′-GGCTCGAG(Xhol) TTTCC GAACTGTGGCTGCACCATCTGTC-3′, and the forward primer 5′-GGGCTAGC (Nhel)TTTCCGAACTGTGGCTGCACCATCTGTC-3′ and the reverse primer 5′-GGCTCGAG(Xhol)CTAACACTCTCCCCTGTTGAAGC-3′ respectively. The CD4D1D2 cDNA fragment is liked to cDNA fragment of IgG2H or IgKLC through restriction enzyme Nhel to create cDNA fragment of CD4D1D2-IgG2HC or CD4D1D2-IgKLC.

e) Construction and transformation of engineered plasmid. The TOPO10 E. coli strain (Invitrogen) is cultured in LB medium containing 500 μg/ml of erythromycin when carrying pJRS233 shuttle vectors. The constructs of ssMIp-T-1249-His, ss-MIp-MIP-1β-His, ssMIp-CD4D1D2-IgG2HC and ssMIp-CD4D1D2-IgKLC is prepared by the insertion of T-1249-His, MIP-1β-His, CD4D1D2-IgG2HC or CD4D1D2-IgKLC cDNA fragments described above, respectively, into MIp-His6 pJRS233 shuttle vector with Pstl and Xhol restriction enzymes. The constructs of BspA-T-1249-His, BspA-MIP-1β-His and BspA-CD4D1D2-IgKLC are prepared through several steps. First, BspA cDNA fragment for addition of an EcoRV restriction enzyme site is produced by PCR from BspA-His6-CFTR pJRS233 shuttle vector with the forward primer 5′-GGTCTAGA(Xbal)ACTTGTTAGTAATGCCG-3′ and the reverse primer 5′-GGGATATC(EcoRv)TTCTGTAATATCCGCACCAAAGT-3′. Second, the T-1249 MIP-1β or CD4D1D2-IgKLC fragments introduced into EcoRV and Pstl two restriction enzymes sites are generated by PCR from ssMIp-T-1249-His, ssMIp-MIP-1β-His and ssMIp-CD4D1D2-IGKLC shuttle vectors with the primers 5′-GGGATATC(EcoRV)TGGCAAGAGTGGGAGCAAAAGATA-3′ and 5′-GGCTGCAG(Pstl)TTAGTGATGATGGTGATGATG-3′, the primers 5′-GGGATATC(EcoRV)TCAGCACCAATGGGCTCAGACC-3′ and 5′-GGCTGCAG(Pstl)TTAGTGATGATGGTGATGATG-3′, and the primers 5′-GGGATATC(EcoRV)ATGAACCGGGGAGTCCCTTTTAGG-3′ and 5′-GGCTGCAG(Pstl)TTAACACTCTCCCCTGTTGAAGC-3′, respectively. Finally the fragment of BspA is linked into the fragments of T-1249, MIp-1β, and CD4D1D1-IgKLC with EcoRV restriction enzyme site respectively, and then inserted into the pJRS233 vector with Xbal and Pstl restriction enzyme sites. For the construct of ssBspA-T-1249-His, ssBspA cDNA fragment for addition of an EcoRV restriction enzyme site is generated by PCR from BspA-His6-CFTR pJRS233 shuttle vector with the primers 5′-GGTCTAGA(Xbal)ACTTGTTAGTAATGCCG-3′ and 5′-GGGATATC(EcoRV)AGATGCCGCATGAATACTGCT-3′, and then the fragment is used to replace BspA fragment in BspA-T-1249-His vector. The constructs of ssSep-T-1249-His, ssSep-MIP-1β-His and ssSep-CD4D1D2-IgKLC are prepared through two steps. First, the cDNA fragment of ssSep introduces an EcoRV restriction enzyme site and is generated by PCR from Sep-His6-Sep pGh:91S S1 plasmid with primers 5′-GGTCTAGA(Xbal)AACCTTCCTGCTGAC-3′ and 5′-is utilized to replace BspA fragment in BspA-T-1249-His, BspA-MIP-1β-His and BspA-CD4D1D2-IgKLC pJRS233 shuttle vector with Xbal and EcoRV restriction enzyme sites respectively. For the construct of ssSep-CD4D1D2-IgKLC, the restriction enzyme sites EcoRV and Pstl is introduced into the CD4D1D2-IgG2HC by PCR from the ssMIp-CD4D1D2-IgG2HC vector using the primer 5′-GGGATATC(EcoRV)ATGAACCGGGGAGTCCCTTTTAGG-3′ and 5′-GGCTGCAG(Pstl)TTATTTACCCGGAGACAGGGAGAG-3′. The resulting fragment is used to replace the CD4D1D2-IgKLC fragment inssSep-CD4D1D2-IgKLC pJRS233 plasmid using the EcoRV and Pstl restriction enzyme sites. The constructed vectors are electrotransformed into Lactobacillus reuteri RC-14 according to the published method Liu et al., using 2.5 μg/ml of penicillin G as a cell wall weakening agent. Lactobacilli is incubated at 30° C. or 40° C. and 5% CO₂ for 3 to 4 days after sublethal incubation at 30° C. and 5% CO₂ overnight.

f) Confirmation of recombinant Lactobacillus reuteri RC-14. The foreign genes integrated into the chromosome of Lactobacillus reuteri RC-14 are confirmed by analysis of PCR products from the genomic DNA and by bacterial cultivation without drug selection (Pozzi, G. et al., 1992, Res. Microbiol., 143: 449-457). Recombinant Lactobacillus reuteri is grown in MRS broth without erythromycin for 36 to 40 hours and then plated in duplicate on MRS agar plates with and without erythromycin, respectively. The genomic DNA of wild-type or recombinant Lactobacillus reuteri RC-14 is prepared described above and utilized to perform nested PCR using the wild type as a negative control. BspA- and ssBspA-constructs, ssMIP-constructs, or ssSep-constructs, three groups of PCR products that are obtained with the forward primer1 5′-CCATTTACAGCCGGCATTATTAC-3′ (corresponding to base to base pairs 443-465 of BspA, gi:2108225) and the nested forward primer 5′-GTAATGGTTGGACCATCTGGATC-3′ (corresponding to base pairs 949-971 of BspA), the forward primer1 5′-CATCCAAGCTTAGTGCGTTAGCC-3′ (corresponding to base pairs 2399-2421 of MIp, gi:32395946) and the nested forward primer 5′-GGCTTGAGCCTTGCTTCTCTTGC-3′ (corresponding to the base pairs 2422-2444 of MIp), or the forward primer1 5′-TGCCGGAAACTACCTATAGTAAGGC-3′ (corresponding to base pairs 29-45 of Sep). BspA-, MIp- and Sep-CD4D1D2-IgG2HC or —CD4D1D2-IgKLC PCR products are with the reverse primer1 5′-CTTTCTTTCCCTGAGTGGCTGCTG-3′ and the nested reverse primer 5′-CCAGTTGCAGCACCAGAAGCAAG-3′. BspA-, MIp- and Sep-MIP-1β PCR products are with the reverse primer 5′-CTACCACAAAGTTGCAGGAAGC-3′ and the nested reverse primer 5′-CCTCGCGGTGTAAGAAAAGCAGC-3′. BspA-, ssBspA-, MIp and Sep-T1249 PCR products are with the reverse primer1 5′-CCAATTCCACAAACTTGCCC-3′ and the nested reverse primer 5′-CTTGTTGGTTCTGCGATTCTTC-3′.

g) Detection of recombinant proteins and peptides. The expression of HIV inhibitor proteins or peptides by recombinant Lactobacillus reuteri RC-14 is determined through Western blotting. For the detection of cell wall-associated proteins, the recombinant Lactobacillus reuteri is cultured in 26 ml of liquid MRS medium to late stationary phase at 37° C. with 5% CO₂. Lactobacillus reuteri is collected by centrifugation at 3500 rpm and 4° C. and washed once with cold PBS. The washed organisms are resuspended in 0.25 ml of PBS and sonicated at 10 W and 30 s for three times (Sonicator W-385, Heat systems). The sonicated bacterial suspension is added with 6×SDS-PAGE loading buffer containing 2.5% β-mercaptoethanol. For the secretion form of recombinant proteins or peptides, the recombinant Lactobacillus reuteri is cultured in MRS to late exponential phase. Twenty-six milliliters of the supernatant is added with 10% TCA and incubated in ice for 3 hours and then centrifuged at 14,000 rpm and 4° C. for 20 minutes. The precipitated proteins or peptides are washed in cold acetone and dissolved in 100 μl of 1×SDS loading buffer including 35 mM NaOH. The cell wall-associated and secreted recombinant proteins or peptides are run at 10% or 15% SDS-PAGE gel and electrotransferred to a nitrocellulose membrane (Bio-Rad) in 10 mM NaHCO₃ and 3 mM Na₂CO₃ transfer buffer (pH 9.9), including 0.037% SDS and 20% methanol. The fusion proteins or peptides are detected by Lumi-light plus Western blotting (Roche) using 0.1 μg ml⁻¹ of monoclonal anti-6 histidine tag antibody (Penta-His antibody, QIAGEN) or 1 μg ml⁻¹ of monoclonal anti-human CD4 antibody (R and D systems) as a primary antibody. The membranes are incubated with the primary antibodies at 4° C. overnight and exposed on X-ray film (X-OMS LS, Kodak) around 1 minute. The concentrations of recombinant proteins are estimated by densitometry on the X-ray films with protein standards using the ImageJ program.

h) In vitro testing of inhibitor activity in tissue culture. The recombinant Lactobacillus reuteri RC-14 strains are cultured to late exponential phase and then collected by centrifugation at 3500 rpm. The supernatant containing secreted Lactobacillus-derived HIV inhibitors are concentrated at 1:150 using an ultra centrifugal filter device (Millipore). The supernatant pH is adjusted to 7.4 and sterilized by filtration. Human PBMCs are prepared as described above. The concentrated supernatant (assay one) is added in the PBMCs (2×10⁵) in a total volume of 120 μl in a 96 well tissue culture plate and incubated at 37° C. and 5% CO₂ for 1 hour. Then, 100 μl of cell-free medium containing TCID₅₀ of HIV-1, 100 TCID₅₀ of SHIV89.6P, or 300 TCID₅₀ of SHIV162PJ virus (The TCID₅₀ is determined by standard method) is added. After 7 days, 120 μl of the cell-free culture medium is collected and used to perform HIV-1 p24 antigen or SIV p27 antigen ELISA following the company protocol (ZeptoMetrix). The antiviral assay (assay two) is also performed, with 100 μl of cell-free medium containing 100 TCID₅₀ of R5 HIV-1 virus incubated with the PBMC for 2 hours in presence of the supernatant. Then, the cells are washed once with PBS and once with the medium. After 7 days, the cell-free medium is collected to perform an ELISA. The viability of PBMC is checked to confirm that the concentrated culture supernatant used for the two assays is be toxic to the cells. The degree of virus inhibition by various recombinant HIV inhibitors is determined through comparison with the wild-type control defined as 100% infection. The statistical significance of the data is determined by Student's t-test.

### Example 3

Viral Production of GALT in Rhesus Macaques.

a) Selection of Animals. It has been demonstrated that Lactobacillus reuteri will colonize the gastrointestinal tracts of infant rhesus macaques safely and effectively (Kelleher, S. L. et al., 2002, Pediatric Gastroenterology and Nutrition, 35(2):162-8). Previous studies analyzing the effects of SIV on the GALT in rhesus macaques is reported by Li et al. (Li, Q. et al., 2005, Nature, 434: 1148-1152). To create statistically reliable results in measuring the effectiveness of the engineered Lactobacillus reuteri in reducing viral load and maintaining pre-infection CD4+T cell levels in the GALT, four rhesus macaques are used in both the control and treatment groups. Three rhesus macaques are treated with PRO 542, MIP-1β, and T-1249 and three rhesus macaques treated with PRO 542 and T-1249 to test whether or not MIP-1β causes significant inflammation in rhesus macaques.

b) Set of Controls. Four randomly selected rhesus macaques are placed in a control group and four other randomly selected rhesus macaques in a treatment group. All of the rhesus macaques from Example 1 one are reused once the probiotics clear from their system. Each rhesus macaque in each group is assigned a number 01-04 for identification. The control group consists of four rhesus macaques that have been intra-vaginally infected with HIV-1 or SHIV but are not orally administered the engineered Lactobacillus reuteri. This will allow for comparison of the treatment group to a group of untreated rhesus macaques undergoing typical viral replication and CD4+T cell depletion in the GALT. The treatment group consists of four rhesus macaques orally administered the engineered Lactobacillus reuteri. CD4+T cell levels in the treatment group are monitored and compared to the results to CD4+T cell depletion of untreated rhesus macaques in the control group to monitor the effectiveness of the treatment intervention.

A special treatment and control group is set up to monitor the effects of administering MIP-1β, which is suspected to cause inflammation. The treatment group contains three rhesus macaques that orally receive Lactobacillus reuteri that secrete PRO 542, MIP-1β, and T-1249. The control group contains three rhesus macaques that receive Lactobacillus reuteri that secretes PRO 542 and T-1249, but not MIP-1β.

c) Oral Administration of Probiotic Lactobacillus reuteri. Microencapsulated Lactobacillus reuteri is administered twice daily with the feeding of the rhesus macaques as described in section Example 1, d).

d) Preparation of HIV-1 and SHIV stocks. The laboratory adapted viruses HIV-1 Bal (R5) and HIV-1 Lai (X4) and several HIV-1 primary isolates (R5a: 91US056 and R5b: 97ZA003; X4a: CMU02 and X4b: CMU10; R5×4a: 92HT593 and R5×4b: 92HT596) are obtained from NIH AIDS Research and Reagent Program. All retroviruses are expanded in PHA (Sigma) stimulated human PBMCs. The PBMCs are cultured in RPMI 1640 medium containing 10% fetal calf serum (Invitrogen) and 50 U of human recombinant interleukin-2 (IL-2) per ml (Roche). The titer of all HIV viral stocks is determined by standard method using human T-cell lines or PBMCs and HIV-1 p24 antigen ELISA (ZeptoMetrix) and are shown to be 10⁴ tissue culture infectious dose per ml (TCID₅₀/ml). SHIV89.6P stock is obtained from Reimann et al. (Reimann, K. A. et al., 1996, J. Virology, 70: 3198-3206) and propagated in macaques PBMCs. The titer of viral stock is determined using MT-2 cells and SIV p27 antigen ELISA (ZeptoMetrix) and shown to be 10⁴ TCID₅₀/ml. SHIV162PJ is isolated from the jejunum of, and originally SHIV162P3 infected, pigtailed macaque. The SHIV162PJ stock is grown in human PBMCs and SIV p27 antigen ELISA and shown to be 10⁴ TCID₅₀/ml.

e) Intra-vaginal Administration. Fourteen adult female rhesus macaques are inoculated intra-vaginally with 2×10⁵ 50% tissue culture infectious dose (TCID₅₀) of HIV-1 or SHIV. A p27 Zeptomertix kit is used to measure the amount of HIV-1 and SHIV cultured in order to titer the stock used to infect the animals. Colon tissue used in the studies from all 14 animals is obtained through needle aspiration and from 2 hours to 28 days after inoculation, as 90% of viral infection of CD4+T cells in the GALT of rhesus macaques takes place 28 days after infection.

f) Measuring CD4+T cell depletion levels in the GALT. CD4+T cell level depletion in the GALT is measured before infection and then during designated points through out the course of treatment to monitor the engineered probiotic's ability to inhibit viral replication in the GALT. Using needle aspiration, CD4+T cell samples are collected from the lamina propria of the control and treatment groups of the rhesus macaques before probiotic administration. This process is repeated once after probiotic administration but before viral infection. Finally, this procedure is carried out every seven days during the course of the treatment (for at least 28 days).

Isolation of the CD4+T cells from the tissue aspirated is started by placing the specimen in a tube containing 10 ml of collagenase digestion solution at 37° C. for 1 hour under constant horizontal shaking at 300 rpm. Placing a 40 μm sieve on a 50 ml tube, the digested sample is transferred under sterile condition through the sieve. The sieve is again washed with 5-10 ml of PBS media and the 50 ml tube is subsequently centrifuged at 300 g and 4° C. for 5 minutes. The supernatant is decanted and the cell pellet is resuspended in 10 ml of ACK lysis buffer. After incubating at 18-25° C. for 5 minutes, the 50 ml tube is centrifuged at 300 g and 4° C. for 5 minutes twice. The supernatant is decanted and the cell pellet washed with 10 ml of PBS media. The centrifugation process is repeated again, and after decanting of the supernatant, the cell pellet is resuspended in 10 ml of RPMI buffer.

After incubation at room temperature, extracellular staining is performed for the identification of CD4+T cells using mAbs (monoclonal antibodies) conjugated with a fluorescent dye (FITC, PE, and APC) (BD, eBiosciences, Biolegend) against the specified markers with corresponding isotope-matched controls (Sigma) (Malkovsky, M. et al., 2003, Clinical and Applied Immunology Reviews. 3:235-245). The cells are then counted and washed using 5 to 10 mL of complete RPMI medium. The cells are then separated into flow tubes, each containing at least five million cells. Surface stain is added to the flow tubes. 10 μL of each stain is added to the flow tubes and incubated for 30 minutes at 4° C.

After initial staining the cells are then washed with PBS (Cellgrow) and fixed with 1% Fixing/Permeabilization (Fix/Perm) buffer (1 mL) (Biolegend). Next, the cells will be vortexed and incubated at room temperature in the dark for 20 minutes (80). After the incubation, cells are again spun down and washed once with PBS. They are then resuspended in 0.1 mL 1% Fixing/Permeabilization (Fix/Perm) buffer. Appropriate amounts of extracellular anti-mAbs (10 μL) are added to each specified tube and then incubated at room temperature for thirty minutes in the dark. After incubation, the cells are washed twice with PBS and resuspended with 0.5 mL of PBS. The cells are now ready for analysis by flow cytometry.

Flow Cytometry is accomplished using BD FACSCalibur Flow Cytometer (BD Bio Sciences) with CellQuest Pro Software. The cytometry results are analyzed using the computer program FlowJo 6.3. In order to determine how many CD4+T cells are present, lymphocytes are displayed on a scatter plot. The physical parameters of the scatter plot are based on the cell's size and granularity. The scatter plot is gated and divided into positive and negative sections for CD4+ and CD4− T cells. This program allows for comparing and contrasting the number of CD4+ and CD4− T cells. By comparing the number of CD4+ and CD4− T cells through out the course of treatment, monitoring of depletion of the level of CD4+T cells is achieved.

g) Evaluation of MIP-1β and inflammation. A complete blood count (CBC) is tabulated to trace for elevated levels of white blood cells in the blood. CBC is a sensitive, but non specific indicator of inflammation and infection. Though Lactobacillus is known to increase CD4+T cell levels, this has only been observed in the GALT and not in systemic blood. However, blood tests are compared between the treatment and control group, such that the control group's baseline white blood cell count incorporates any elevation due to the presence of Lactobacillus in the gut. Endoscopy is used to visually look for sites of inflammation in the gastrointestinal mucosal tissue.

In conclusion, by establishing the ability of engineered Lactobacillus reuteri RC-14 to colonize in the gastrointestinal tracts of rhesus macaques, secrete PRO 542, MIP-1β, and T-1249, and prevent or reduce CD4+T cell depletion in the GALT, this engineered Lactobacillus reuteri RC-14 is useful as a probiotic prophylaxis to slow or prevent initial infection of HIV in humans. This treatment may also be useful to slow the progression of an already existing HIV infection, and to prevent the development of AIDS. Because of the three-stage approach to blocking HIV from replicating, the likelihood of developing drug resistance during treatment in later stages of infection is decreased. As such, an HIV-positive infected patient's quality of life for a longer period of time can be achieved.

The invention and the manner and process of making and using it are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims.

What is claimed is:

1. A method for treating or preventing a Human Immunodeficiency Virus (HIV) infection, or treating or preventing Acquired Human Immunodeficiency Syndrome (AIDS), in a subject in need thereof, comprising:
a) engineering Lactobacillus reuteri RC-14 to secrete at least two fusion inhibitors;
b) colonizing the engineered probiotic Lactobacillus reuteri RC-14 in the gastrointestinal tract of the subject;
c) secreting the fusion inhibitors in the gastrointestinal tract; and
d) decreasing or preventing HIV production and CD4+T cell depletion in the gastrointestinal tract because of the presence of the two or more fusion inhibitors, wherein the two or more fusion inhibitors are selected from the group consisting of PRO 542, MIP-1β and T-1249.

2. A method according to claim 1 wherein the fusion inhibitors are PRO 542 and T-1249.

3. A method according to claim 2 wherein the fusion inhibitors additionally include MIP-1β.

4. A method according to claim 1 wherein colonization of the engineered probiotic Lactobacillus reuteri RC-14 in the gastrointestinal tract of the subject is achieved by oral administration of the engineered probiotic Lactobacillus reuteri RC-14 to the subject.

5. A method of reducing or preventing HIV replication in a CD4+T cell in gut associated lymphatic tissue (GALT) of a gastrointestinal tract, comprising interfering with the binding, fusion or entry of HIV viron into the CD4+T cell in GALT by two or more fusion inhibitors secreted by a genetically modified probiotic Lactobacillus reuteri RC-14.

6. A method according to claim 5 wherein two or more stages of HIV viral entry are reduced or blocked.

7. A method according to claim 6 wherein the two or more fusion inhibitors are selected from the group consisting of PRO 542, MIP-1β and T-1249.

8. A method according to claim 7 wherein the fusion inhibitors are all three of PRO 542, MIP-1β and T-1249 and three stages of HIV viral entry are reduced or blocked.

9. A method according to claim 5 wherein presence of the engineered probiotic Lactobacillus reuteri RC-14 in the gastrointestinal tract is achieved by oral administration of the engineered probiotic Lactobacillus reuteri RC-14.

10. A method of reducing or preventing depletion of CD4+T cells in GALT of a gastrointestinal tract caused by initial HIV infection, comprising reducing or preventing HIV replication in the GALT by presence of two or more fusion inhibitors secreted by a genetically modified probiotic Lactobacillus reuteri RC-14.

11. A method according to claim 10 wherein the two or more fusion inhibitors are selected from the group consisting of PRO 542, MIP-1β and T-1249.

12. A method according to claim 11 wherein the fusion inhibitors are all three of PRO 542, MIP-1β and T-1249.

13. A method according to claim 10 wherein the presence of the engineered probiotic Lactobacillus reuteri RC-14 is achieved by oral administration of the engineered probiotic Lactobacillus reuteri RC-14.

14. A method according to claim 5 wherein the interfering with the binding, fusion or entry of HIV viron into the CD4+T cell in GALT by two or more fusion inhibitors comprises two or all three of a) inhibiting CCR5, b) preventing binding to CD4, and c) interfering with spring-loaded action of viral gp41 and hairpin formation.


SEQUENCE LISTING (40 sequences)

SEQ ID NO: 1 - DNA, 23 bp, Artificial Sequence
  Synthetic DNA sequence
tattgctgacttgcaacaagcac

SEQ ID NO: 2 - DNA, 22 bp, Artificial Sequence
  Synthetic DNA sequence
ttcttcttgctgtaagcgtacc

SEQ ID NO: 3 - DNA, 23 bp, Artificial Sequence
  Synthetic DNA sequence
ttcgtcaataaagcctaacaagg

SEQ ID NO: 4 - DNA, 23 bp, Artificial Sequence
  Synthetic DNA sequence
ctaatacggtagtcactaatagc

SEQ ID NO: 5 - DNA, 19 bp, Artificial Sequence
  Synthetic DNA sequence
gtaaccttcctgctgacct

SEQ ID NO: 6 - DNA, 22 bp, Artificial Sequence
  Synthetic DNA sequence
caacaccctatatactacactc

SEQ ID NO: 7 - DNA, 68 bp, Artificial Sequence
  Synthetic DNA sequence
ggctgcaggaactgcctcagcatggcaagagtgggagcaaaagatatacagcttaattgaagaatcgc

SEQ ID NO: 8 - DNA, 52 bp, Artificial Sequence
  Synthetic DNA sequence
gcctcgagttagtgatgatggtgatgatgaaaccaattccacaaacttgccc

SEQ ID NO: 9 - DNA, 44 bp, Artificial Sequence
  Synthetic DNA sequence
gcctgcaggaactgcctcagcatcagcaccaatgggctcagacc

SEQ ID NO: 10 - DNA, 51 bp, Artificial Sequence
  Synthetic DNA sequence
ggctcgagttagtgatgatggtgatgatggttcagttccaggtcatacacg

SEQ ID NO: 11 - DNA, 46 bp, Artificial Sequence
  Synthetic DNA sequence
ggctgcaggaactgcctcagcaatgaaccggggagtcccttttagg

SEQ ID NO: 12 - DNA, 24 bp, Artificial Sequence
  Synthetic DNA sequence
ctggaaagctagcaccacgatgtc

SEQ ID NO: 13 - DNA, 35 bp, Artificial Sequence
  Synthetic DNA sequence
gggctagctttcgagccaaatgttgtgtcgagtgc

SEQ ID NO: 14 - DNA, 36 bp, Artificial Sequence
  Synthetic DNA sequence
ggctcgagtttccgaactgtggctgcaccatctgtc

SEQ ID NO: 15 - DNA, 36 bp, Artificial Sequence
  Synthetic DNA sequence
gggctagctttccgaactgtggctgcaccatctgtc

SEQ ID NO: 16 - DNA, 31 bp, Artificial Sequence
  Synthetic DNA sequence
ggctcgagctaacactctcccctgttgaagc

SEQ ID NO: 17 - DNA, 25 bp, Artificial Sequence
  Synthetic DNA sequence
ggtctagaacttgttagtaatgccg

SEQ ID NO: 18 - DNA, 31 bp, Artificial Sequence
  Synthetic DNA sequence
gggatatcttctgtaatatccgcaccaaagt

SEQ ID NO: 19 - DNA, 32 bp, Artificial Sequence
  Synthetic DNA sequence
gggatatctggcaagagtgggagcaaaagata

SEQ ID NO: 20 - DNA, 29 bp, Artificial Sequence
  Synthetic DNA sequence
ggctgcagttagtgatgatggtgatgatg

SEQ ID NO: 21 - DNA, 30 bp, Artificial Sequence
  Synthetic DNA sequence
gggatatctcagcaccaatgggctcagacc

SEQ ID NO: 22 - DNA, 29 bp, Artificial Sequence
  Synthetic DNA sequence
ggctgcagttagtgatgatggtgatgatg

SEQ ID NO: 23 - DNA, 32 bp, Artificial Sequence
  Synthetic DNA sequence
gggatatcatgaaccggggagtcccttttagg

SEQ ID NO: 24 - DNA, 31 bp, Artificial Sequence
  Synthetic DNA sequence
ggctgcagttaacactctcccctgttgaagc

SEQ ID NO: 25 - DNA, 25 bp, Artificial Sequence
  Synthetic DNA sequence
ggtctagaacttgttagtaatgccg

SEQ ID NO: 26 - DNA, 29 bp, Artificial Sequence
  Synthetic DNA sequence
gggatatcagatgccgcatgaatactgct

SEQ ID NO: 27 - DNA, 23 bp, Artificial Sequence
  Synthetic DNA sequence
ggtctagaaaccttcctgctgac

SEQ ID NO: 28 - DNA, 32 bp, Artificial Sequence; identical to SEQ ID NO: 23
  Synthetic DNA sequence

SEQ ID NO: 29 - DNA, 32 bp, Artificial Sequence
  Synthetic DNA sequence
ggctgcagttatttacccggagacagggagag

SEQ ID NO: 30 - DNA, 23 bp, Artificial Sequence
  Synthetic DNA sequence
ccatttacagccggcattattac

SEQ ID NO: 31 - DNA, 23 bp, Artificial Sequence
  Synthetic DNA sequence
gtaatggttggaccatctggatc

SEQ ID NO: 32 - DNA, 23 bp, Artificial Sequence
  Synthetic DNA sequence
catccaagcttagtgcgttagcc

SEQ ID NO: 33 - DNA, 23 bp, Artificial Sequence
  Synthetic DNA sequence
ggcttgagccttgcttctcttgc

SEQ ID NO: 34 - DNA, 25 bp, Artificial Sequence
  Synthetic DNA sequence
tgccggaaactacctatagtaaggc

SEQ ID NO: 35 - DNA, 24 bp, Artificial Sequence
  Synthetic DNA sequence
ctttctttccctgagtggctgctg

SEQ ID NO: 36 - DNA, 23 bp, Artificial Sequence
  Synthetic DNA sequence
ccagttgcagcaccagaagcaag

SEQ ID NO: 37 - DNA, 22 bp, Artificial Sequence
  Synthetic DNA sequence
ctaccacaaagttgcaggaagc

SEQ ID NO: 38 - DNA, 23 bp, Artificial Sequence
  Synthetic DNA sequence
cctcgcggtgtaagaaaagcagc

SEQ ID NO: 39 - DNA, 20 bp, Artificial Sequence
  Synthetic DNA sequence
ccaattccacaaacttgccc

SEQ ID NO: 40 - DNA, 22 bp, Artificial Sequence
  Synthetic DNA sequence
cttgttggttctgcgattcttc